(19) United States
(12) Patent Application Publication
(10) Pub. No.: US 2024/0269517 A1
(43) Pub. Date: Aug. 15, 2024

(54) Aqueous Propulsion Devices For Prostheses

(71) Applicant: Timothy Michael Walsh, Cocoa Beach, FL (US)

(72) Inventor: Timothy Michael Walsh, Cocoa Beach, FL (US)

(21) Appl. No.: 18/429,497

(22) Filed: Feb. 1, 2024

Related U.S. Application Data
(60) Provisional application No. 63/576,403, filed on Feb. 9, 2023.

(51) Int. Cl. A63B 31/12; A61F 2/60; A61F 2/50

(52) U.S. Cl. CPC A63B 31/12; A61F 2/60; A61F 2002/5089; A61F 2002/607

(57) ABSTRACT

The present disclosure provides aqueous propulsion devices for prostheses. An aqueous propulsion device may comprise at least one substantially flat protruding extension member configured to extend from an outer surface of a lower-limb prosthesis. In some aspects, the aqueous propulsion device may be configured to be at least temporarily secured to a lower-limb prosthesis via one or more attachment mechanisms. In some implementations, the aqueous propulsion device may comprise a plurality of protruding extension members. In some embodiments, the aqueous propulsion device may comprise two protruding extension members configured on opposing lateral sides of the aqueous propulsion device. In some aspects, securing the aqueous propulsion device to a lower-limb prosthesis may enable a user to swim or otherwise move through a body of water by exerting a pushing force on the water that propels the user in a desired direction when the user kicks or engages in a similar movement.

### CROSS-REFERENCE TO RELATED APPLICATION

This application is a Non-provisional of and claims priority to U.S. Provisional Application Ser. No. 63/576,403 (filed Feb. 9, 2023 and titled “PROSTHETIC LOWER-LIMB ACCESSORY FOR SWIMMING”), the entire contents of which are incorporated herein by reference.

### BACKGROUND

Human mobility is a necessary component of everyday life that is often taken for granted. Walking, running, swimming, and similar activities are needed for transportation, exercise, or recreation. In instances where an individual's limbs are afflicted with severe pain, disease, or injury, the ability to move around can be significantly hindered, if not altogether halted. In such circumstances, the affected limb(s) may be surgically removed and replaced with a prosthetic limb. While prostheses greatly improve the ability of a person to move around comfortably, they do not always restore full mobility in all scenarios.

One challenge faced by those who utilize lower-limb prostheses is moving effectively in water. In fact, many currently available lower-limb prostheses are not even waterproof, and those that are fail to enable a user to swim through the water with a satisfactory amount of propulsion due to the rigidity and relatively small lateral profile of the prosthesis. This, unfortunately, forces individuals to forgo an enjoyable and highly-effective form of recreational enjoyment and exercise, thereby contributing to a decreased overall quality of life.

### SUMMARY OF THE DISCLOSURE

What is needed is a device that facilitates the ability of users of lower-limb prostheses to swim or otherwise move in water or similar fluid. In some aspects, an aqueous propulsion device is disclosed that comprises at least one protruding extension member and at least one attachment mechanism, wherein the attachment mechanism may be configured to at least temporarily secure the protruding extension member(s) to at least one portion of a lower-limb prosthesis.

A number of embodiments of the present disclosure will be described. While this specification contains many specific implementation details, these should not be construed as limitations on the scope of any disclosures or of what may be claimed, but rather as descriptions of features specific to particular embodiments of the present disclosure. It is understood to those skilled in the art that variations, modifications, and alterations may be apparent. It will be understood that various modifications may be made without departing from the spirit and scope of the disclosure.

### BRIEF DESCRIPTION OF THE DRAWINGS

The accompanying drawings that are incorporated in and constitute a part of this specification illustrate several embodiments of the disclosure and, together with the description, serve to explain the principles of the disclosure:

FIG. 1 illustrates a perspective view of an exemplary aqueous propulsion device, according to some embodiments of the present disclosure.

FIG. 2 illustrates a side view of an exemplary attachment mechanism for an aqueous propulsion device, according to some embodiments of the present disclosure.

FIG. 3 illustrates a top view of an exemplary aqueous propulsion device, according to some embodiments of the present disclosure.

FIG. 4 illustrates a front view of an exemplary aqueous propulsion device, according to some embodiments of the present disclosure.

FIG. 5 illustrates an exemplary aqueous propulsion device, according to some embodiments of the present disclosure.

FIG. 6 illustrates a front view of an exemplary aqueous propulsion device, according to some embodiments of the present disclosure.

FIG. 7 illustrates a perspective view of an exemplary aqueous propulsion device, according to some embodiments of the present disclosure.

FIG. 8A illustrates a front view of an exemplary aqueous propulsion device, according to some embodiments of the present disclosure.

FIG. 8B illustrates a front view of an exemplary aqueous propulsion device, according to some embodiments of the present disclosure.

FIG. 9A illustrates a front view of an exemplary aqueous propulsion device, according to some embodiments of the present disclosure.

FIG. 9B illustrates a front view of an exemplary aqueous propulsion device, according to some embodiments of the present disclosure.

FIG. 10A illustrates a front view of an exemplary aqueous propulsion device, according to some embodiments of the present disclosure.

FIG. 10B illustrates a front view of an exemplary aqueous propulsion device, according to some embodiments of the present disclosure.

FIG. 11A illustrates a front view of an exemplary aqueous propulsion device, according to some embodiments of the present disclosure.

FIG. 11B illustrates a front view of an exemplary aqueous propulsion device, according to some embodiments of the present disclosure.

FIG. 11C illustrates a front view of an exemplary aqueous propulsion device, according to some embodiments of the present disclosure.

FIG. 12A illustrates a front view of an exemplary aqueous propulsion device, according to some embodiments of the present disclosure.

FIG. 12B illustrates a front view of an exemplary aqueous propulsion device, according to some embodiments of the present disclosure.

FIG. 12C illustrates a front view of an exemplary aqueous propulsion device, according to some embodiments of the present disclosure.

FIG. 12D illustrates a front view of an exemplary aqueous propulsion device, according to some embodiments of the present disclosure.

FIG. 13A illustrates a front view of an exemplary aqueous propulsion device, according to some embodiments of the present disclosure.

FIG. 13B illustrates a front view of an exemplary aqueous propulsion device, according to some embodiments of the present disclosure.

### DETAILED DESCRIPTION

The present disclosure provides generally for aqueous propulsion devices for prostheses. According to the present disclosure, an aqueous propulsion device is disclosed that may be at least temporarily secured to at least one portion of a lower-limb prosthesis such that the aqueous propulsion device may assist a user with swimming or otherwise moving through a body of water when the aqueous propulsion device is secured to the lower-limb prosthesis and the lower-limb prosthesis is worn by the user.

In some aspects, an aqueous propulsion device in accordance with the present disclosure may be configured to be removably secured to at least one portion of a lower-limb prosthesis via one or more attachment mechanisms. In some implementations, the aqueous propulsion device may be configured to be secured without the use of any tools.

In some embodiments, an aqueous propulsion device may comprise at least one substantially flat or planar extension member configured to protrude outwardly from the outer surface of the lower-limb prosthesis when the aqueous propulsion device is secured thereto. In some aspects, each protruding extension member may comprise a “fin” or “wing” shape with an at least partially curved or actuate top and/or bottom portion. In some embodiments, each protruding extension member may comprise a rigidity sufficient to exert a pushing force on an amount of water or a similar fluid when a user engages in a kicking or similar motion to propel the user through the water in an intended direction.

In some implementations, an aqueous propulsion device may be secured to a user's lower-limb prosthesis to assist with the user's mobility in water. In some embodiments, the aqueous propulsion device may comprise a plurality of protruding extension members. By way of example and not limitation, an aqueous propulsion device may comprise two protruding extension members, with each protruding extension member configured on an opposing lateral side of the lower-limb prosthesis relative to the other protruding extension member.

In the following sections, detailed descriptions of examples and methods of the disclosure will be given. The descriptions of both preferred and alternative examples, though thorough, are exemplary only, and it is understood to those skilled in the art that variations, modifications, and alterations may be apparent. It is therefore to be understood that the examples do not limit the broadness of the aspects of the underlying disclosure as defined by the claims.

### Glossary

Lower-limb prosthesis: as used herein refers to any artificial device or component configured to replace at least a portion of a user's leg, such as the foot, ankle, and/or shin, as non-limiting examples.
Aqueous: as used herein refers to water or any similar liquid or fluid through which a human or animal may travel or otherwise move.

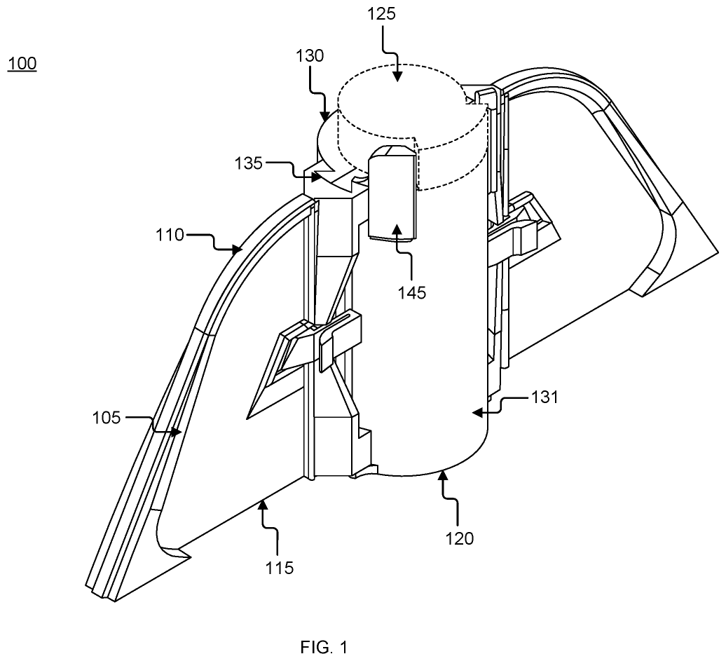

Referring now to FIG. 1, a perspective view of an exemplary aqueous propulsion device 100, according to some embodiments of the present disclosure, is illustrated. In some aspects, the aqueous propulsion device 100 may comprise at least one protruding extension member 105. In some implementations, each protruding extension member 105 may comprise a substantially flat or planar configuration. In some non-limiting exemplary embodiments, each protruding extension member 105 may comprise an at least partially curved or arcuate top portion 110. In some implementations, a bottom portion 115 of each protruding extension member 105 may be substantially flat, though other structural configurations are considered within the scope of the present disclosure. In some aspects, each protruding extension member 105 may comprise a “fin,” “wing,” or similar structural configuration.

In some aspects, the aqueous propulsion device 100 may comprise a plurality of protruding extension members 105. In some implementations, by way of example and not limitation, the aqueous propulsion device 100 may comprise two protruding extension members 105, wherein the protruding extension members 105 may be configured on opposing lateral sides of the aqueous propulsion device 100. In some embodiments, each protruding extension member 105 may at least partially comprise one or more substantially rigid and/or one or more at least partially flexible, pliable, or elastic materials, such as metal, plastic, rubber, or carbon fiber, as non-limiting examples.

In some implementations, the aqueous propulsion device 100 may comprise at least one attachment mechanism, such as, for example and not limitation, at least one cylindrical component 120. In some aspects, an inner surface of the cylindrical component 120 may be configured to physically abut an outer surface of at least one portion of a lower-limb prosthesis 125 when the aqueous propulsion device 100 is at least temporarily secured to the lower-limb prosthesis 125. In some non-limiting exemplary embodiments, the cylindrical component 120 may comprise two semicylindrical portions 130, 131, wherein the semicylindrical portions 130, 131 may be configured to be removably secured to each other to at least temporarily secure the aqueous propulsion device 100 to the lower-limb prosthesis 125.

In some aspects, each semicylindrical portion 130, 131 may comprise at least one projecting element 135 configured to extend outwardly from an outer surface thereof. In some embodiments, each protruding extension member 105 may comprise a proximal surface configured to be adjacent to the cylindrical component 120 and/or the lower-limb prosthesis when at least temporarily secured thereto, wherein at least one portion of the proximal surface may comprise at least one recess, channel, or groove. In some implementations, the recesses of each protruding extension member 105 may be configured to slidably receive corresponding projecting elements 135 of the semicylindrical portions 130, 131, such that the semicylindrical portions 130, 131 may be at least temporarily secured together when received by the recesses of the protruding extension members 105 to form the cylindrical component 120 around the lower-limb prosthesis 125.

In some embodiments, at least one of the semicylindrical portions 130, 131 may comprise at least one alignment element 145. In some implementations, the alignment element 145 may comprise a physical extension of the structure of one or both of the semicylindrical portions 130, 131 that may force a user to align the semicylindrical portions 130, 131 and attach the protruding extension members 105 in the correct orientation to ensure a stable configuration of the aqueous propulsion device 100 when at least temporarily secured to the lower-limb prosthesis 125. In some aspects, this may minimize the likelihood of the aqueous propulsion device 100 from coming apart unintentionally or from experiencing premature mechanical failure, as non-limiting examples. In some embodiments, each alignment element 145 may be configured to physically interface with at least one portion of the lower-limb prosthesis 125 to minimize or prevent unintentional or undesired rotation of the cylindrical component 120 around the lower-limb prosthesis 125.

In some implementations, the semicylindrical portions 130, 131 may be configurable in one or more of a plurality of sizes, shapes, or designs to accommodate a plurality of sizes, configurations, shapes, designs, or styles of different lower-limb prostheses 125. In some aspects, this may allow the aqueous propulsion device 100 to be used with a variety of different lower-limb prostheses 125, depending on user needs or preferences. In some non-limiting exemplary embodiments, the semicylindrical portions 130, 131 may be manufactured to comprise different sizes or configurations, or the semicylindrical portions 130, 131 may comprise one or more adjustable elements that may allow a single set of semicylindrical portions 130, 131 to be at least temporarily secured to lower-limb prostheses 125 of different sizes or structural designs. By way of example and not limitation, the semicylindrical portions 130, 131 may comprise one or more extendable or retractable portions that may enable the width or height of one or both of the semicylindrical portions 130, 131 to be increased or decreased, wherein one or more detents or similar catches may lock the relevant semicylindrical portion 130, 131 at a required or preferred size until unlocked by at least one release mechanism, such as a button or lever, as non-limiting examples.

In some non-limiting exemplary embodiments, the aqueous propulsion device 100 may be configured to be used with an upper-limb prosthesis, such as an arm, wrist, or hand, as non-limiting examples. In some implementations, this may facilitate the ability of a user to move through water or similar fluid using one or more arm motions.

In some aspects, the lower-limb prosthesis 125 may be configured to be at least partially submerged in water or a similar fluid. By way of example and not limitation, the lower limb-prosthesis 125 may comprise one or more lightweight, water resistant, waterproof, or antimicrobial materials. In some non-limiting exemplary embodiments, the lower-limb prosthesis 125 may comprise one or more apertures and/or pumping mechanisms to facilitate removal or drainage of water as needed or desired.

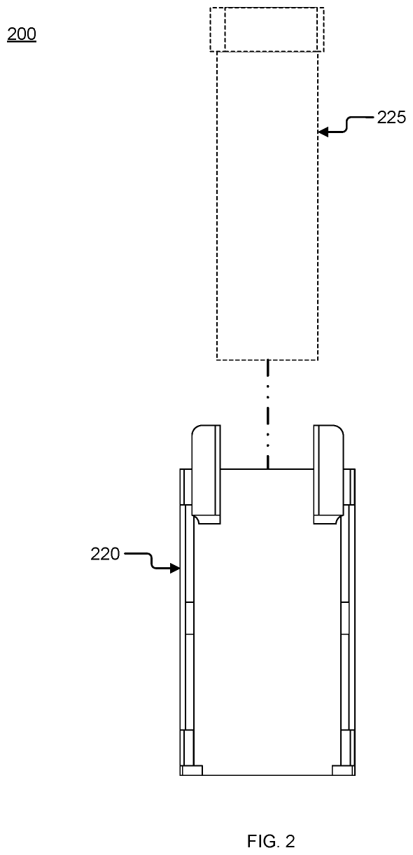

Referring now to FIG. 2, a side view of an exemplary attachment mechanism for an aqueous propulsion device 200, according to some embodiments of the present disclosure, is illustrated. In some aspects, two semicylindrical portions may be configured to form an attachment mechanism in the form of a cylindrical component 220 that may be configured to be at least temporarily secured to at least one portion of a lower-limb prosthesis 225. In some implementations, an inner surface of the cylindrical component 220 may be configured to physically abut at least one portion of an outer surface of the lower-limb prosthesis 225 when the cylindrical component 220 is at least temporarily secured thereto.

In some aspects, the cylindrical component 220 formed by the semicylindrical portions may comprise an attachment mechanism that forms a base structure that may be at least temporarily secured to the lower-limb prosthesis 225 and to which one or more protruding extension members may be at least temporarily secured. In some non-limiting exemplary embodiments, the protruding extension members may comprise “fins,” “wings,” or similar structural designs that may assist a user with swimming or otherwise moving through a body of water. In some implementations, the protruding extension members may comprise any configuration, shape, or design that may assist a user with movement, comfort, or in completing one or more tasks, as non-limiting examples. By way of example and not limitation, the protruding extension members may at least partially comprise floatation devices, comfort padding, or exercise facilitating devices or equipment, as non-limiting examples.

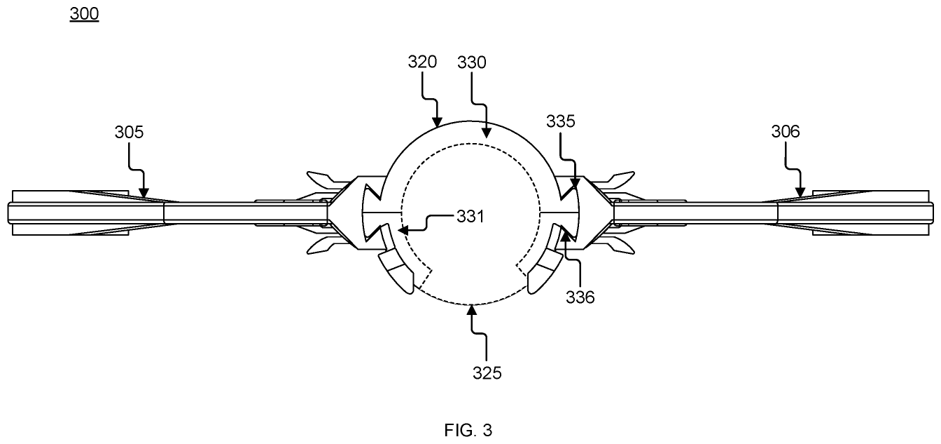

Referring now to FIG. 3, a top view of an exemplary aqueous propulsion device 300, according to some embodiments of the present disclosure, is illustrated. In some aspects, the aqueous propulsion device 300 may be configured to be at least temporarily secured to at least one portion of a lower-limb prosthesis 325. In some implementations, the aqueous propulsion device 300 may comprise at least one attachment mechanism in the form of at least one cylindrical component 320. In some embodiments, the cylindrical component 320 may be configured to be formed when two semicylindrical portions 330, 331 are at least temporarily secured together around a portion of a lower-limb prosthesis 325, wherein an inner surface of each semicylindrical portion 330, 321 may be configured to physically abut an outer surface of the lower-limb prosthesis when the cylindrical component 320 is at least temporarily secured thereto.

In some implementations, each semicylindrical portion 330, 331 may comprise at least one projecting element 335, 336 configured to extend from an outer surface thereof. In some embodiments, corresponding projecting elements 335, 336 of each semicylindrical portion 330, 331 may be configured to be physically aligned when the semicylindrical portions 330, 331 are placed laterally adjacent to each other to form the cylindrical component 320, such that the projecting elements 335, 336 may be slidably received by one or more recesses, channels, or grooves configured at, within, or near a proximal surface of each protruding extension member 305, 306 to at least temporarily secure the semicylindrical portions 330, 331 around the lower-limb prosthesis 325 to form the aqueous propulsion device 300.

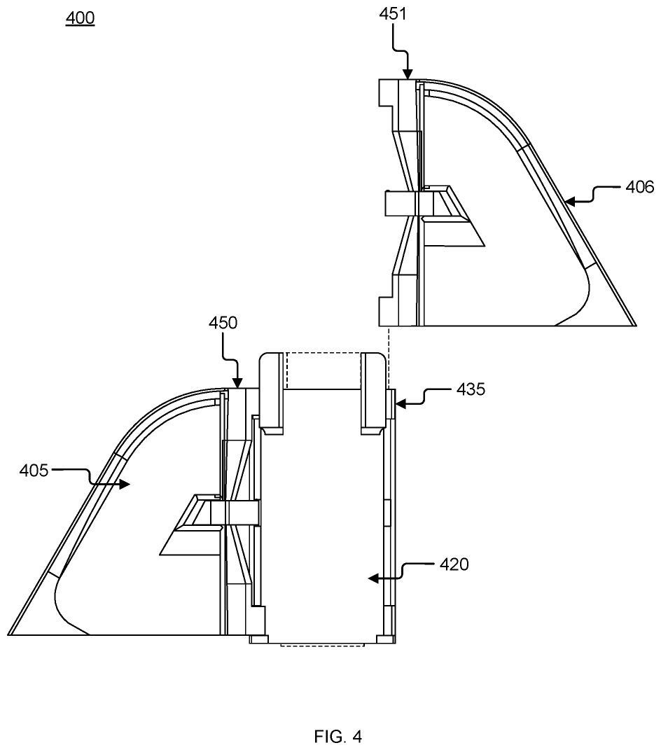

Referring now to FIG. 4, a front view of an exemplary aqueous propulsion device 400, according to some embodiments of the present disclosure, is illustrated. In some aspects, the aqueous propulsion device 400 may comprise at least one attachment mechanism in the form of at least one cylindrical component 420. In some implementations, the cylindrical component 420 may be configured to be at least temporarily secured to at least one portion of a lower-limb prosthesis, wherein an inner surface of the cylindrical component 420 may be configured to physically abut an outer surface of the lower-limb prosthesis when the cylindrical component 420 is at least temporarily secured thereto.

In some non-limiting exemplary embodiments, the cylindrical component 420 may comprise one or more projecting elements 435, such as tabs, ridges, or similar structures, that may extend from an outer surface of the cylindrical component 420. In some implementations, the projecting elements 435 may be configured to be slidably received by one or more recesses 440 configured within one or more channels or grooves within or upon a proximal surface 450, 451 of one or more protruding extension members 405, 406. In some aspects, the proximal surface 450, 451 of each protruding extension member 405, 406 may be configured to be adjacent to the outer surface of the cylindrical component 420 when the protruding extension members 405, 406 are at least temporarily secured thereto.

In some non-limiting exemplary implementations, the cylindrical component 420 may comprise two semicylindrical portions. In some embodiments, each semicylindrical portion may comprise at least one projecting element 435, such that corresponding projecting elements 435 of each semicylindrical portion may be adjacently aligned when the semicylindrical portions are combined to form the cylindrical component 420. In some aspects, the semicylindrical portions may be at least temporarily secured together around at least one portion of a lower-limb prosthesis when the aligned projecting elements 435 are slidably received by one or more recesses 440 of each protruding extension member 405, 406.

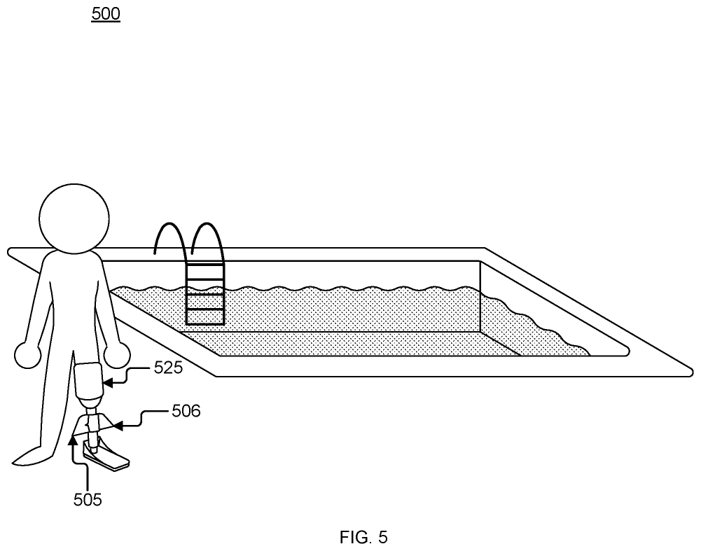

Referring now to FIG. 5, an exemplary aqueous propulsion device 500, according to some embodiments of the present disclosure, is illustrated. In some aspects, the aqueous propulsion device 500 may be configured to be at least temporarily secured to at least one portion of a lower-limb prosthesis 525. In some implementations, the aqueous propulsion device 500 may comprise at least one protruding extension member 505, 506 configured to extend outwardly from an outer surface of the lower-limb prosthesis 525. In some embodiments, the aqueous propulsion device 500 may be at least temporarily secured to at least one portion of the lower-limb prosthesis 525 via at least one attachment mechanism.

In some non-limiting exemplary implementations, the aqueous propulsion device 500 may comprise one or more protruding extension members 505, 506 in the form of substantially flat or substantially planar structures, such as “fins” or “wings,” as non-limiting examples. In some aspects, each protruding extension member 505, 506 may be configured to exert a pushing force upon an amount of water or similar fluid that propels a user in an intended direction when the user engages in a kicking motion or similar movement, thereby helping he user swim or otherwise move through the water, such as for exercise or recreation, as non-limiting examples. In some non-limiting exemplary embodiments, the aqueous propulsion device 500 may comprise two protruding extension members 505, 506 configured on opposing lateral sides of the aqueous propulsion device 500, wherein the size, shape, and orientation of the protruding extension members 505, 506 may be configured to approximately mimic the forces applied by a human foot while engaging in a kicking motion while swimming, as a non-limiting example.

Referring now to FIG. 6, a front view of an exemplary aqueous propulsion device 600, according to some embodiments of the present disclosure, is illustrated. In some aspects, the aqueous propulsion device 600 may be configured to be at least temporarily secured to at least one portion of a lower-limb prosthesis 625 via at least one attachment mechanism. In some implementations, the aqueous propulsion device 600 may comprise one or more protruding extension members 605, 606, 607, 608, wherein each protruding extension member 605, 606, 607, 608 may be configured to extend from an outer surface of the lower-limb prosthesis 625. In some aspects, each protruding extension member 605, 606, 607, 608 may comprise a substantially flat or substantially planar structural configuration, such as a “fin” or “wing” design, as non-limiting examples.

In some non-limiting exemplary embodiments, the aqueous propulsion device 600 may comprise a plurality of protruding extension members 605, 606, 607, 608. By way of example and not limitation, the aqueous propulsion device 600 may comprise multiple pairs of protruding extension members 605, 606, 607, 608, wherein each pair of protruding extension members 605, 606, 607, 608 may comprise two protruding extension members 605, 606, 607, 608 configured on opposing lateral sides of the aqueous propulsion device 600. In some aspects, this configuration may increase the amount of force applied by the aqueous propulsion device 600 when a user engages in a kicking or similar motion, thereby increasing the ease and/or speed with which the user may move through a body of water or similar fluid, as non-limiting examples.

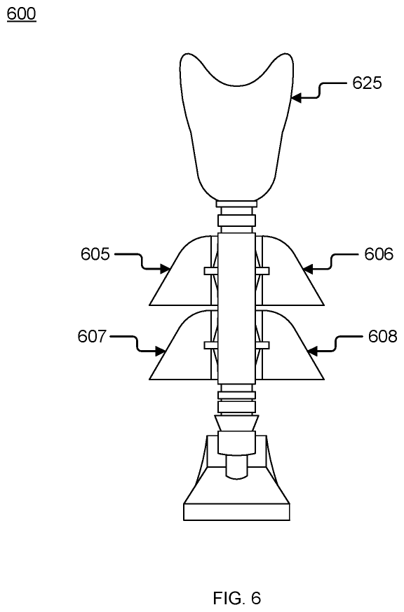

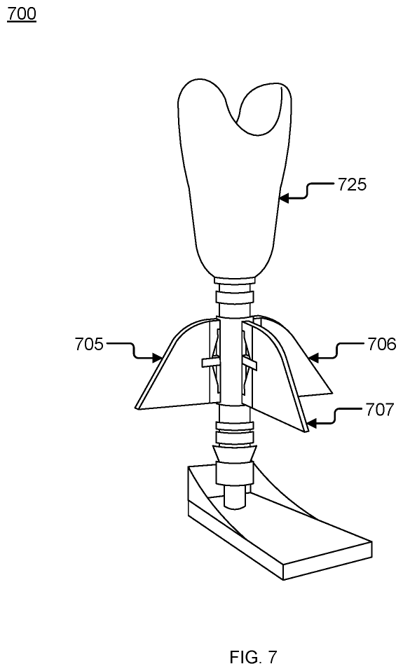

Referring now to FIG. 7, a perspective view of an exemplary aqueous propulsion device 700, according to some embodiments of the present disclosure, is illustrated. In some aspects, the aqueous propulsion device 700 may be configured to be at least temporarily secured to at least one portion of a lower-limb prosthesis 725 via at least one attachment mechanism. In some implementations, the aqueous propulsion device 700 may comprise one or more protruding extension members 705, 706, 707, wherein each protruding extension member 705, 706, 707 may be configured to extend from an outer surface of the lower-limb prosthesis 725. In some aspects, each protruding extension member 705, 706, 707 may comprise a substantially flat or substantially planar structural configuration, such as a “fin” or “wing” design, as non-limiting examples.

In some non-limiting exemplary embodiments, the aqueous propulsion device 700 may comprise a plurality of protruding extension members 705, 706, 707. By way of example and not limitation, the aqueous propulsion device 700 may comprise two protruding extension members 705, 706 configured on opposing lateral sides of the aqueous propulsion device 700, as well as a protruding extension member 707 configured on a front portion of the aqueous propulsion device 700. In some implementations, the laterally opposing protruding extension members 705, 706 may be configured to exert a pushing force on an amount of water or similar fluid that propels a user in an intended direction when the user engages in a kicking or similar movement, while the protruding extension member 707 at the front of the aqueous propulsion device 700 may be configured to reduce the drag forces and minimize the resistance associated therewith when the user moves the lower-limb prosthesis 725 forward through fluid, such as while swimming or walking, as non-limiting examples.

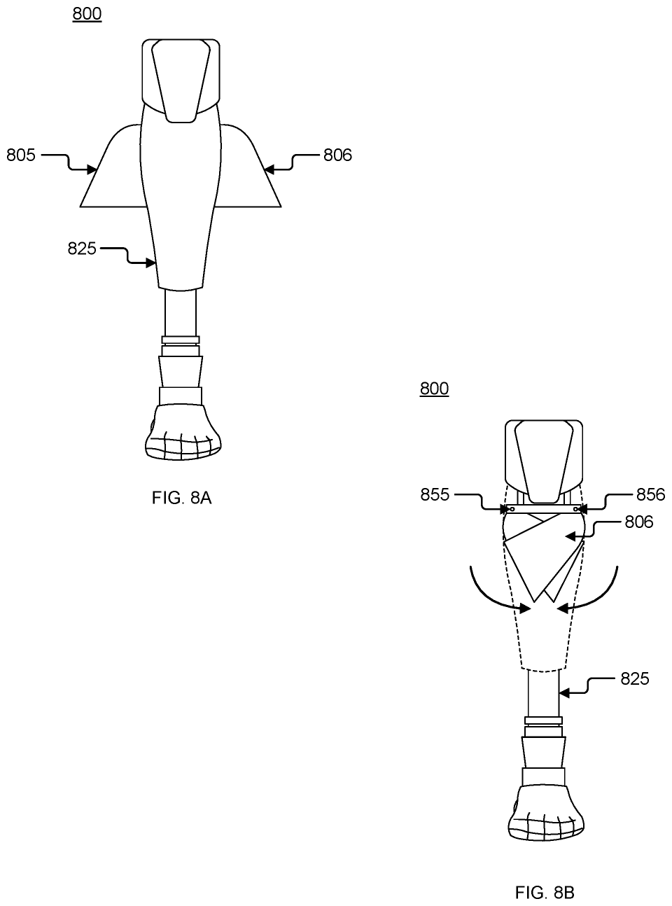

Referring now to FIGS. 8A-8B, front views of an exemplary aqueous propulsion device 800, according to some embodiments of the present disclosure, are illustrated. In some aspects, the aqueous propulsion device 800 may be configured to be at least temporarily secured to at least one portion of a lower-limb prosthesis 825 via at least one attachment mechanism. In some implementations, the aqueous propulsion device 800 may comprise one or more protruding extension members 805, 806, wherein each protruding extension member 805, 806 may be configured to extend outwardly relative to an outer surface of the lower-limb prosthesis 825. In some aspects, each protruding extension member 805, 806 may comprise a substantially flat or substantially planar structural configuration, such as a “fin” or “wing” design, as non-limiting examples.

In some non-limiting exemplary embodiments, the aqueous propulsion device 800 may be secured to or integrated with the lower-limb prosthesis 825 via one or more attachment mechanisms in the form of bolts, screws, adhesives, nails, pins, or similar minimally-removable fastening elements. In some implementations, the protruding extension members 805, 806 may be configured to be alterable between an extended position and a retracted position. In some aspects, the protruding extension members 805, 806 may be fully exposed when in the extended position, such that they may be used to exert a pushing force on an amount of water or similar fluid when a user engages in a kicking or similar movement, thereby propelling the user in an intended direction and facilitating the ability of the user to swim or otherwise move through the fluid, as non-limiting examples. In some embodiments, the protruding extension members 805, 806 may be configured to be completely contained within one or more internal portions of the lower-limb prosthesis 825, such as, for example and not limitation, slots, chambers, or similar receptacles, when in the retracted position, thereby allowing the user to use the lower-limb prosthesis 825 in a non-aquatic environment comfortably.

In some non-limiting exemplary implementations, the aqueous propulsion device 800 may comprise two protruding extension members 805, 806, wherein the protruding extension members 805, 806 may be configured on opposing lateral sides of the aqueous propulsion device 800. In some embodiments, each protruding extension member 805, 806 may be configured to be alterable between the extended position and the retracted position by pivoting or rotating about at least one hinge 855, 856. In some aspects, each protruding extension member 805, 806 may be configured to interact with at least one spring mechanism, such as a compression spring, an extension spring, or a torsion spring, as non-limiting examples, wherein the spring mechanism may be configured to exert one or more forces upon the associated protruding extension member 805, 806 that may bias the protruding extension member 805, 806 towards the extended position or the retracted position.

In some aspects, each protruding extension member 805, 806 may be configured to interact with at least one securing mechanism, wherein the securing mechanism may be configured to lock the associated protruding extension member 805, 806 in the extended position and/or the retracted position. In some implementations, the securing mechanism may be configured to be altered from a locked state to an unlocked state by at least one release mechanism, wherein the release mechanism may be configured to be actuated by a user to unlock and release the associated protruding extension member 805, 806 from the locked extended or locked retracted position. By way of example and not limitation, the securing mechanism may comprise one or more detents or similar catches that may be securely received by one or more notches or similar recesses when in the locked state, wherein the release mechanism may be configured to physically remove the detent(s) from the recess(es) to unlock and release the securing mechanism when the release mechanism is activated by a user.

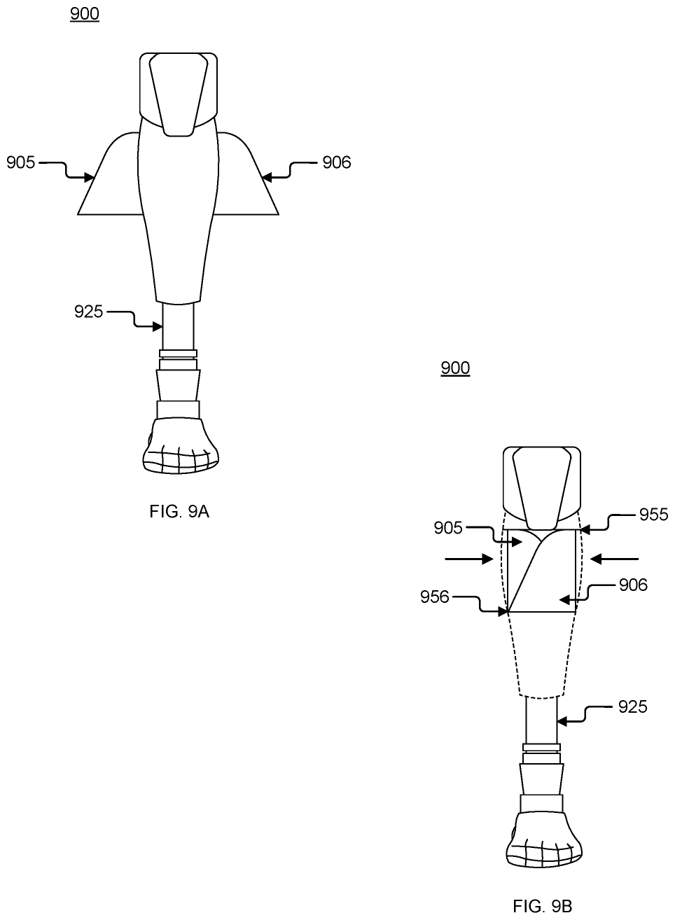

Referring now to FIGS. 9A-9B, front views of an exemplary aqueous propulsion device 900, according to some embodiments of the present disclosure, are illustrated. In some aspects, the aqueous propulsion device 900 may be configured to be at least temporarily secured to at least one portion of a lower-limb prosthesis 925 via at least one attachment mechanism. In some implementations, the aqueous propulsion device 900 may comprise one or more protruding extension members 905, 906, wherein each protruding extension member 905, 906 may be configured to extend outwardly relative to an outer surface of the lower-limb prosthesis 925. In some aspects, each protruding extension member 905, 906 may comprise a substantially flat or substantially planar structural configuration, such as a “fin” or “wing” design, as non-limiting examples.

In some non-limiting exemplary embodiments, the aqueous propulsion device 900 may be secured to or integrated with the lower-limb prosthesis 925 via one or more attachment mechanisms in the form of bolts, screws, adhesives, nails, pins, or similar minimally-removable fastening elements. In some implementations, the protruding extension members 905, 906 may be configured to be alterable between an extended position and a retracted position. In some aspects, the protruding extension members 905, 906 may be fully exposed when in the extended position, such that they may be used to exert a pushing force on an amount of water or similar fluid when a user engages in a kicking or similar movement, thereby propelling the user in an intended direction and facilitating the ability of the user to swim or otherwise move through the fluid, as non-limiting examples. In some embodiments, the protruding extension members 905, 906 may be configured to be completely contained within one or more internal portions of the lower-limb prosthesis 925, such as, for example and not limitation, slots, chambers, or similar receptacles, when in the retracted position, thereby allowing the user to use the lower-limb prosthesis 925 in a non-aquatic environment comfortably.

In some non-limiting exemplary implementations, the aqueous propulsion device 900 may comprise two protruding extension members 905, 906, wherein the protruding extension members 905, 906 may be configured on opposing lateral sides of the aqueous propulsion device 900. In some embodiments, each protruding extension member 905, 906 may be configured to be alterable between the extended position and the retracted position by transversely sliding along or within at least one track 955, 956 or similar channel or groove. In some aspects, each protruding extension member 905, 906 may be configured to interact with at least one spring mechanism, such as a compression spring or an extension spring, as non-limiting examples, wherein the spring mechanism may be configured to exert one or more forces upon the associated protruding extension member 905, 906 that may bias the protruding extension member 905, 906 towards the extended position or the retracted position.

In some aspects, each protruding extension member 905, 906 may be configured to interact with at least one securing mechanism, wherein the securing mechanism may be configured to lock the associated protruding extension member 905, 906 in the extended position and/or the retracted position. In some implementations, the securing mechanism may be configured to be altered from a locked state to an unlocked state by at least one release mechanism, wherein the release mechanism may be configured to be actuated by a user to unlock and release the associated protruding extension member 905, 906 from the locked extended or locked retracted position. By way of example and not limitation, the securing mechanism may comprise one or more detents or similar catches that may be securely received by one or more notches or similar recesses when in the locked state, wherein the release mechanism may be configured to physically remove the detent(s) from the recess(es) to unlock and release the securing mechanism when the release mechanism is actuated by a user.

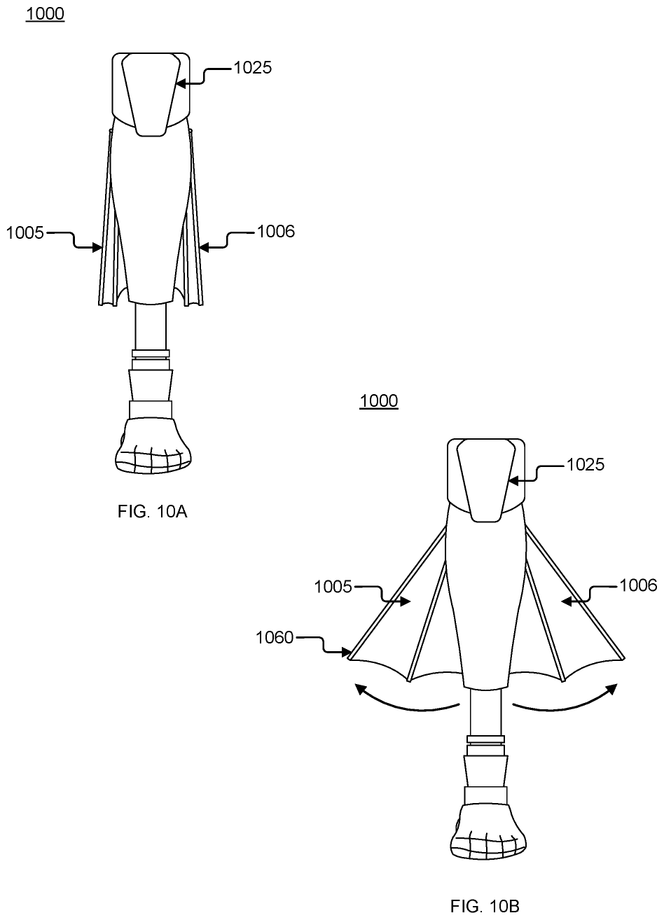

Referring now to FIGS. 10A-10B, front views of an exemplary aqueous propulsion device 1000, according to some embodiments of the present disclosure, are illustrated. In some aspects, the aqueous propulsion device 1000 may be configured to be at least temporarily secured to at least one portion of a lower-limb prosthesis 1025 via at least one attachment mechanism. In some implementations, the aqueous propulsion device 1000 may comprise one or more protruding extension members 1005, 1006, wherein each protruding extension member 1005, 1006 may be configured to extend outwardly relative to an outer surface of the lower-limb prosthesis 1025. In some aspects, each protruding extension member 1005, 1006 may comprise a substantially flat or substantially planar structural configuration, such as a “fin” or “wing” design, as non-limiting examples.

In some non-limiting exemplary embodiments, the aqueous propulsion device 1000 may be secured to or integrated with the lower-limb prosthesis 1025 via one or more attachment mechanisms in the form of bolts, screws, adhesives, nails, pins, or similar minimally-removable fastening elements. In some implementations, the protruding extension members 1005, 1006 may be configured to be alterable between an extended position and a retracted position. In some aspects, the protruding extension members 1005, 1006 may be fully exposed when in the extended position, such that they may be used to exert a pushing force on an amount of water or similar fluid when a user engages in a kicking or similar movement, thereby propelling the user in an intended direction and facilitating the ability of the user to swim or otherwise move through the fluid, as non-limiting examples. In some embodiments, the protruding extension members 1005, 1006 may be configured to comprise a collapsed structure when in the retracted position, thereby allowing the user to use the lower-limb prosthesis 1025 in a non-aquatic environment comfortably with a minimal profile extending beyond the outer perimeter of the lower-limb prosthesis 1025.

In some non-limiting exemplary implementations, the aqueous propulsion device 1000 may comprise two protruding extension members 1005, 1006, wherein the protruding extension members 1005, 1006 may be configured on opposing lateral sides of the aqueous propulsion device 1000. In some embodiments, an upper portion of each protruding extension member 1005, 1006 may be configured to facilitate altering of each protruding extension member 1005, 1006 between the extended position and the retracted position by pivoting or rotating about at least one hinge. In some aspects, each protruding extension member 1005, 1006 may be configured to interact with at least one spring mechanism, such as a compression spring, an extension spring, or a torsion spring, as non-limiting examples, wherein the spring mechanism may be configured to exert one or more forces upon the associated protruding extension member 1005, 1006 that may bias the protruding extension member 1005, 1006 towards the extended position or the retracted position.

In some aspects, each protruding extension member 1005, 1006 may be configured to interact with at least one securing mechanism, wherein the securing mechanism may be configured to lock the associated protruding extension member 1005, 1006 in the extended position and/or the retracted position. In some implementations, the securing mechanism may be configured to be altered from a locked state to an unlocked state by at least one release mechanism, wherein the release mechanism may be configured to be actuated by a user to unlock and release the associated protruding extension member 1005, 1006 from the locked extended or locked retracted position. By way of example and not limitation, the securing mechanism may comprise one or more detents or similar catches that may be securely received by one or more notches or similar recesses when in the locked state, wherein the release mechanism may be configured to physically remove the detent(s) from the recess(es) to unlock and release the securing mechanism when the release mechanism is actuated by a user.

In some non-limiting exemplary embodiments, each protruding extension member 1005, 1006 may at least partially comprise one or more foldable or collapsible materials, such as nylon, polyester, or pongee, as non-limiting examples. In some aspects, this may allow the protruding extension members 1005, 1006 to achieve a minimal physical profile when in the retracted position. In some implementations, each protruding extension member 1005, 1006 may comprise one or more substantially rigid members 1060, such as, for example and not limitation, bands, bars, or ribs, to add structural integrity to the protruding extension member 1005, 1006 when in the extended position. By way of example and not limitation, each substantially rigid member 1060 may comprise one or more substantially or sufficiently rigid materials, such as hard plastic, wood, or rubber, as non-limiting examples.

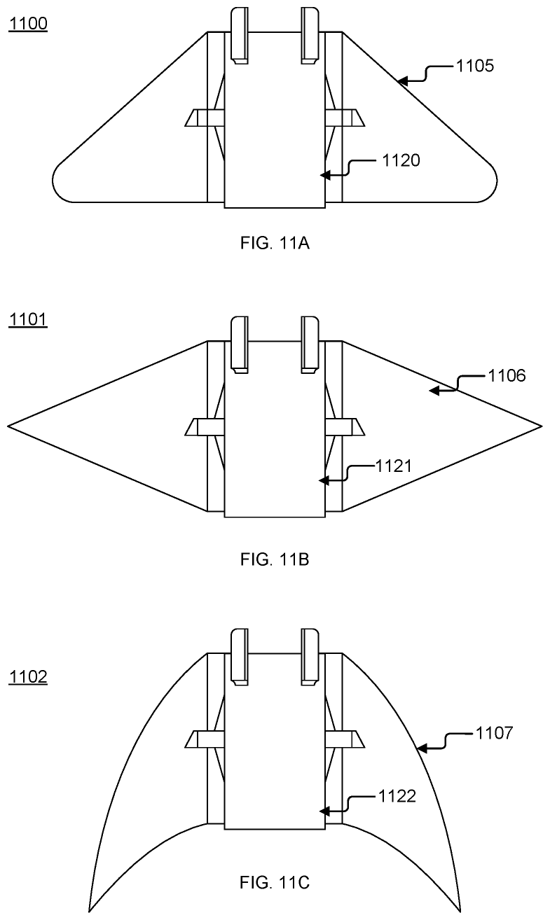

Referring now to FIGS. 11A-11C, front views of exemplary aqueous propulsion devices 1100, 1101, 1102, according to some embodiments of the present disclosure, are illustrated. In some aspects, the aqueous propulsion device 1100, 1101, 1102 may be configured to be at least temporarily secured to at least one portion of a lower-limb prosthesis via at least one attachment mechanism, such as, for example and not limitation, at least one cylindrical component 1120, 1121, 1122. In some implementations, the aqueous propulsion device 1100, 1101, 1102 may comprise one or more protruding extension members 1105, 1106, 1107, wherein each protruding extension member 1105, 1106, 1107 may be configured to extend outwardly relative to an outer surface of the lower-limb prosthesis. In some aspects, each protruding extension member 1105, 1106, 1107 may comprise a substantially flat or substantially planar structural configuration, such as a “fin” or “wing” design, as non-limiting examples. In some implementations, the cylindrical component 1120, 1121, 1122 may be configured to be at least temporarily secured to at least one portion of a lower-limb prosthesis, wherein an inner surface of the cylindrical component 1120, 1121, 1122 may be configured to physically abut an outer surface of the lower-limb prosthesis when the cylindrical component 1120, 1121, 1122 is at least temporarily secured thereto.

In some non-limiting exemplary embodiments, the cylindrical component 1120, 1121, 1122 may comprise one or more projecting elements, such as tabs, ridges, or similar structures, that may extend from an outer surface of the cylindrical component 1120, 1121, 1122. In some implementations, the projecting elements may be configured to be slidably received by one or more recesses configured within one or more channels or grooves within or upon a proximal surface of one or more protruding extension members 1105, 1106, 1107. In some aspects, the proximal surface of each protruding extension member 1105, 1106, 1107 may be configured to be adjacent to the outer surface of the cylindrical component 1120, 1121, 1122 when the protruding extension members 1105, 1106, 1107 are at least temporarily secured thereto.

In some implementations, the protruding extension member(s) 1105, 1106, 1107 of the aqueous propulsion device 1100, 1101, 1102 may each comprise one or more of a plurality of shapes, designs, or configurations. By way of example and not limitation, the protruding extension member 1105 may comprise a triangular shape with a rounded corner and a bottom portion that is substantially parallel with the bottom of the cylindrical component 1120, the protruding extension member 1106 may comprise a triangular shape with a pointer corner with substantially flat top and bottom portions that are angled or slanted relative to the top and bottom of the cylindrical component 1121, and the protruding extension member 1107 may comprise curved or arcuate top and bottom portions, as non-limiting examples.

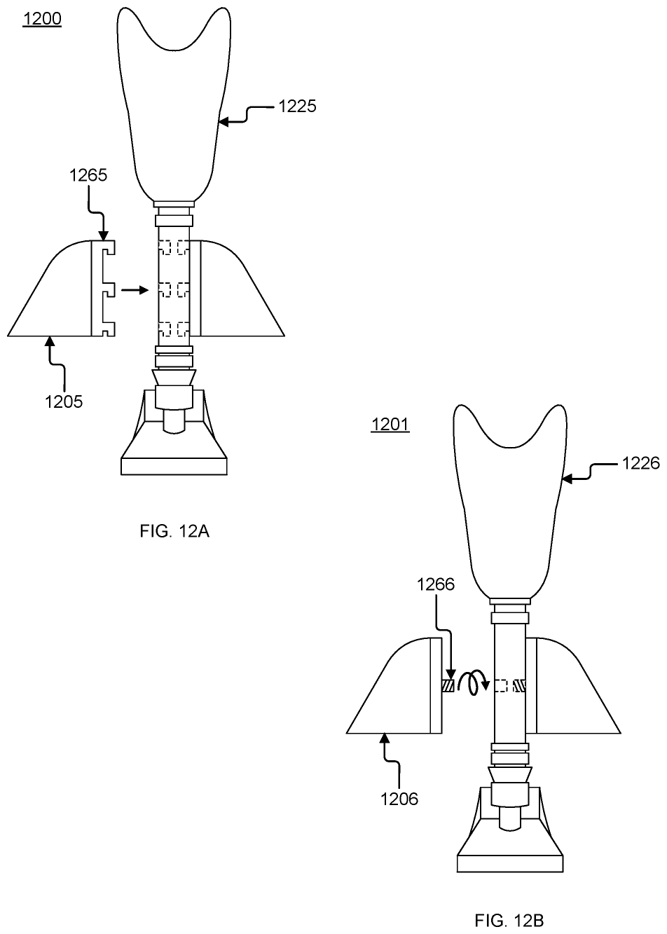

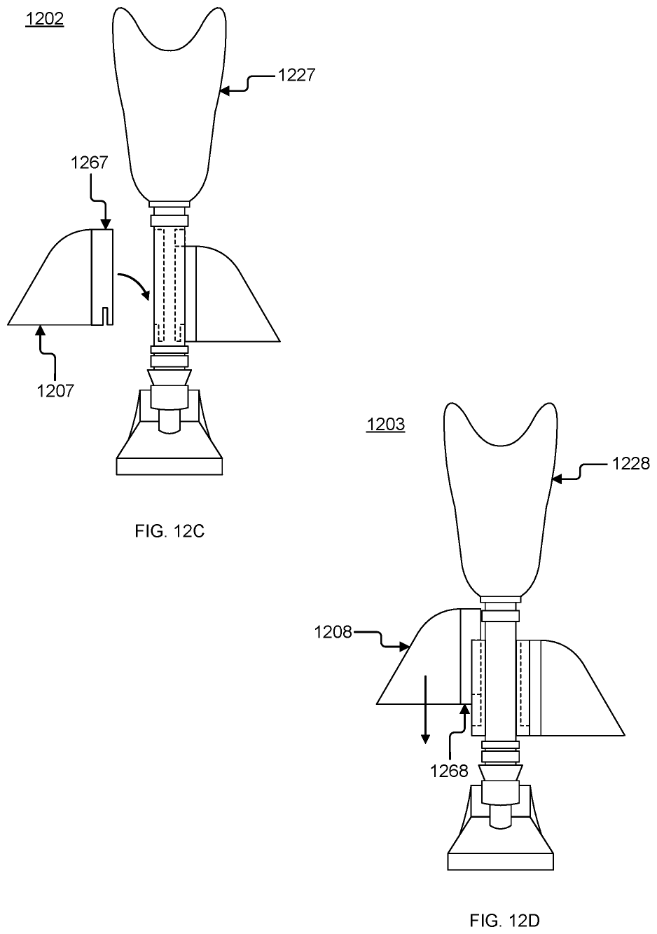

Referring now to FIGS. 12A-12D, front views of an exemplary aqueous propulsion device 1200, 1201, 1203, 1203, according to some embodiments of the present disclosure, are illustrated. In some aspects, the aqueous propulsion device 1200, 1201, 1202, 1203 may comprise one or more protruding extension members 1205, 1206, 1207, 1208 that may be configured to be at least temporarily secured to at least one portion of a lower-limb prosthesis 1225, 1226, 1227, 1228 via at least one attachment mechanism 1265, 1266, 1267, 1268. In some implementations, each protruding extension member 1205, 1206, 1207, 1208 may be configured to extend from an outer surface of the lower-limb prosthesis 1225, 1226, 1227, 1228 when at least temporarily secured thereto. In some non-limiting exemplary embodiments, each protruding extension member 1205, 1206, 1207, 1208 may comprise a substantially flat or substantially planar structural configuration, such as a “fin” or “wing” design, as non-limiting examples.

In some non-limiting exemplary implementations, the aqueous propulsion device 1200, 1201, 1202, 1203 may comprise two protruding extension members 1205, 1206, 1207, 1208 configured to be removably secured to opposing lateral sides of the lower-limb prosthesis 1225, 1226, 1227, 1228. In some aspects, this configuration may enable a user to exert one or more pushing forces on an amount of water or similar fluid via the protruding extension members 1205, 1206, 1207, 1208 when the user engages in a kicking or similar motion, thereby propelling the user through the water in an intended direction.

In some non-limiting exemplary embodiments, each protruding extension member 1205, 1206, 1207, 1208 may be at least temporarily secured to at least one portion of the lower-limb prosthesis 1225, 1226, 1227, 1228 via one or more attachment mechanisms 1265, 1266, 1267, 1268. In some implementations, each attachment mechanism 1265, 1266, 1267, 1268 may comprise corresponding elements configured upon or within the each protruding extension member 1205, 1206, 1207, 1208 and the lower-limb prosthesis 1225, 1226, 1227, 1228. By way of example and not limitation, the protruding extension member 1205 may comprise one or more prongs, tabs, or similar protruding attachment mechanisms 1265 that may be at least temporarily received within one or more corresponding holes, notches, or recesses within the lower-limb prosthesis 1225. In some embodiments, the attachment mechanism(s) 1265 may be configured to snap into the corresponding recess(es) within the lower-limb prosthesis 1225 for added security or stability.

As an additional non-limiting illustrative example, the protruding extension member 1206 may comprise one or more attachment mechanisms 1266 in the form of male threaded screws that may be at least temporarily received within one or more corresponding female threaded recesses within the lower-limb prosthesis 1226. By way of further example and not limitation, the protruding extension member 1207 may comprise an attachment mechanism 1267 in the form of an elongated ridge that may be received within a corresponding slot, channel, or groove within the lower-limb prosthesis 1227. In some non-limiting exemplary embodiments, the end of the ridge of the protruding extension member 1207 may comprise an extension portion configured to fit within a corresponding notch within the lower-limb prosthesis 1227 to secure the protruding extension member 1207 in place and facilitate prevention of unintentional release of the protruding extension member 1207.

As a further non-limiting illustrative example, the protruding extension member 1208 may comprise an attachment mechanism 1268 in the form of an elongated ridge or tab that may be slidably received within a slotted or recessed structure configured upon an outer surface of the lower-limb prosthesis 1228. In some aspects, the attachment mechanism 1268 may be configured to maintain its position within the recessed structure via a snug friction fit.

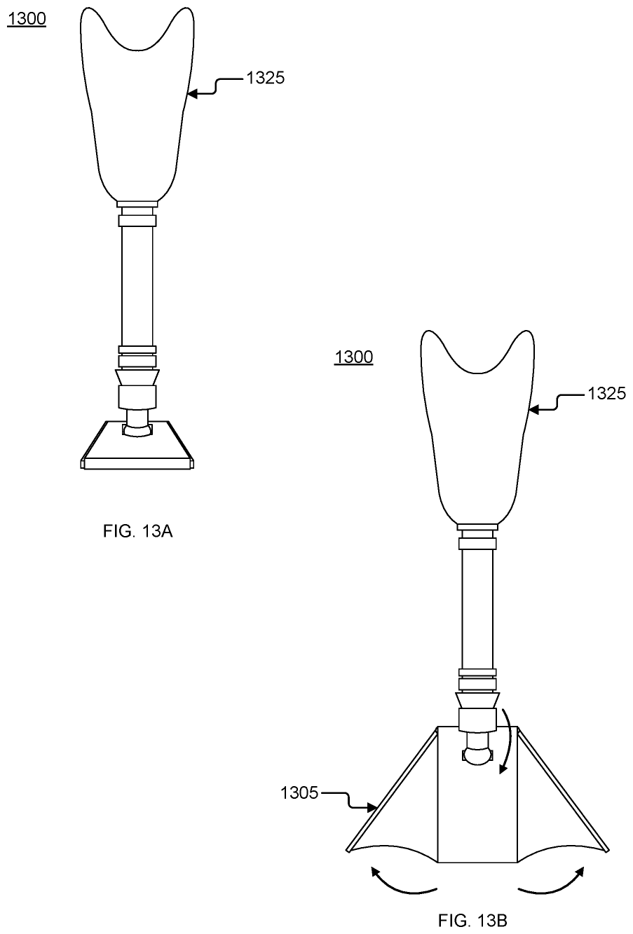

Referring now to FIGS. 13A-13B, front views of an exemplary aqueous propulsion device 1300, according to some embodiments of the present disclosure, are illustrated. In some non-limiting illustrative embodiments, the aqueous propulsion device 1300 may be integrated with a lower-limb prosthesis 1325. In some implementations, the bottom portion of the lower-limb prosthesis 1325 may comprise an artificial “foot” portion that may be dislodged or released from a first position that may facilitate ambulatory movement of a user, and rotated or pivoted to a second position via rotation about at least one hinge, wherein a plane comprising the “foot” portion may be substantially parallel to the longitudinal axis of the lower-limb prosthesis when in the second position. In some aspects, the “foot” portion may be locked or snapped in to the second position using at least one securing mechanism to prevent or minimize unintentional movement of the “foot” portion, such as, for example and not limitation, by using one or more detents or similar catches.

In some implementations, once the “foot” portion of the lower-limb prosthesis 1325 is in the second position, one or more protruding extension members 1305 may be altered from a retracted position within one or more internal portions of the “foot,” such as, for example and not limitation, slots, chambers, or similar receptacles, to an extended position, wherein the protruding extension member(s) 1305 may be fully exposed when in the extended position, such that they may be used to exert a pushing force on an amount of water or similar fluid when a user engages in a kicking or similar movement, thereby propelling the user in an intended direction and facilitating the ability of the user to swim or otherwise move through the fluid, as non-limiting examples. In some embodiments, each protruding extension member 1305 may comprise a substantially flat or substantially planar structural configuration, such as a “fin” or “wing” design, as non-limiting examples. In some implementations, the protruding extension member(s) 1305 may be configured to be locked into place when in the extended or retracted position, such as, for example and not limitation, via one or more detents or similar catches, wherein at least one release mechanism may be configured to be actuated by the user to unlock and release the protruding extension member(s) as needed.

In some aspects, by configuring the “foot” portion of the lower-limb-prosthesis to convert from an ambulatory position to an aqueous propulsion device 1300, a user may be able to experience maximum utility of two different use configurations using a single apparatus. In some implementations, this may enhance user convenience and efficiency, as non-limiting examples.

### CONCLUSION

A number of embodiments of the present disclosure have been described. While this specification contains many specific implementation details, these should not be construed as limitations on the scope of any disclosures or of what may be claimed, but rather as descriptions of features specific to particular embodiments of the present disclosure.

Certain features that are described in this specification in the context of separate embodiments can also be implemented in combination or in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Moreover, the separation of various system components in the embodiments described above should not be understood as requiring such separation in all embodiments, and it should be understood that the described program components and systems can generally be integrated together in a single product or packaged into multiple products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order show, or sequential order, to achieve desirable results. In certain implementations, multitasking and parallel processing may be advantageous. Nevertheless, it will be understood that various modifications may be made without departing from the spirit and scope of the claimed disclosure.

What is claimed is:

1. An aqueous propulsion device for a lower-limb prosthesis, comprising:
at least one protruding extension member; and
at least one attachment mechanism, wherein the at least one attachment mechanism is configured to at least temporarily secure the at least one protruding extension member to at least one portion of the lower-limb prosthesis.

2. The aqueous propulsion device of claim 1, wherein the at least one protruding extension member comprises a substantially flat configuration.

3. The aqueous propulsion device of claim 1, wherein a top portion of the protruding extension member comprises an arcuate configuration.

4. The aqueous propulsion device of claim 1, wherein a bottom portion of the protruding extension member comprises a substantially flat configuration.

5. The aqueous propulsion device of claim 1, wherein the aqueous propulsion device comprises a plurality of the at least one protruding extension member.

6. The aqueous propulsion device of claim 5, wherein the plurality of the at least one protruding extension member comprises a first at least one protruding extension member and a second at least one protruding extension member.

7. The aqueous propulsion device of claim 6, wherein the first at least one protruding extension member and the second at least one protruding extension member are configured on opposing lateral sides of the aqueous propulsion device.

8. The aqueous propulsion device of claim 1, wherein the at least one attachment mechanism comprises a cylindrical component, wherein an interior surface of the cylindrical component is configured to abut an outer surface of the lower-limb prosthesis when the aqueous propulsion device is at least temporarily secured to the lower-limb prosthesis.

9. The aqueous propulsion device of claim 8, wherein the cylindrical component comprises a first semicylindrical portion and a second semicylindrical portion.

10. The aqueous propulsion device of claim 9, wherein at least one portion of a proximal surface of the at least one protruding extension member adjacent to the outer surface of the lower-limb prosthesis comprises at least one recess, wherein the at least one recess is configured to slidably receive one or more projecting elements extending from an outer surface of each of the first semicylindrical portion and the second semicylindrical portion.

11. The aqueous propulsion device of claim 1, wherein the at least one protruding extension member comprises one or more at least partially flexible materials.

12. The aqueous propulsion device of claim 1, wherein the at least one protruding extension member is configured to be altered between an extended position and a retracted position.

13. The aqueous propulsion device of claim 12, wherein the at least one protruding extension member is configured to be completely contained within at least one internal portion of the lower-limb prosthesis when in the retracted position.

14. The aqueous propulsion device of claim 12, wherein the at least one protruding extension member is configured to be altered between the extended position and the retracted position by rotating about at least one hinge.

15. The aqueous propulsion device of claim 12, wherein the at least one protruding extension member is configured to be altered between the extended position and the retracted position by sliding along at least one track.

16. The aqueous propulsion device of claim 12, wherein the at least one protruding extension member is configured to interact with at least one spring mechanism.

17. The aqueous propulsion device of claim 16, wherein the at least one spring mechanism is configured to bias the at least one protruding extension member towards one or both of: the retracted position or the extended position.

18. The aqueous propulsion device of claim 16, wherein the at least one spring mechanism comprises at least one of: a torsion spring, a compression spring, and an extension spring.

19. The aqueous propulsion device of claim 12, wherein the at least one protruding extension member at least partially comprises at least one collapsible material, wherein the at least one protruding extension member comprises a collapsed configuration when in the retracted position.

20. The aqueous propulsion device of claim 1, wherein the at least one protruding extension member comprises one or more substantially rigid materials.
